(19) United States
(12) Patent Application Publication
(10) Pub. No.: US 2016/0279393 A1
(43) Pub. Date: Sep. 29, 2016

(54) GUIDEWIRE RETRIEVAL CATHETER

(71) Applicant: Covidien LP, Mansfield, MA (US)

(72) Inventors: Christopher Anderson, Plymouth, MN (US); Bradley Steele, Plymouth, MN (US)

(21) Appl. No.: 14/668,005

(22) Filed: Mar. 25, 2015

(51) Int. Cl. A61M 25/09

(52) U.S. Cl. CPC A61M 25/09041

(57) ABSTRACT

A guidewire retrieval catheter includes a guidewire retrieval lumen extending lengthwise of a catheter body. An inflatable member at the distal end of the catheter body defines an inflation chamber for selectively receiving fluid to configure the inflatable member from deflated configuration to an inflated configuration. The inflatable member has an inner wall defining a guidewire retrieval cavity in communication with the guidewire retrieval lumen to facilitate insertion of the guidewire into the guidewire retrieval lumen when the inflatable member is in the inflated configuration. The guidewire retrieval catheter is used to retrieve a guidewire in a body lumen.

### FIELD OF THE DISCLOSURE

The present disclosure generally relates to a guidewire retrieval catheter for retrieving a guidewire disposed within a body lumen.

### BACKGROUND OF THE DISCLOSURE

In some medical procedures, it may be necessary to retrieve a guidewire disposed within a body lumen. In particular, in some cases a guidewire may need to be advanced through a body lumen in a direction that is opposite that of the advancement of the medical device used with the guidewire. Introducing the guidewire in such a fashion is a retrograde procedure. For example, a guidewire may be introduced in a retrograde procedure when attempting to re-vascularize a chronic total occlusion (CTO) of an arterial vessel. In such a procedure, it may be difficult to penetrate a proximal cap of the CTO with the guidewire. Accordingly, a tip of the guidewire is introduced at distal location relative to the CTO so that the tip of the guidewire can penetrate a softer distal cap of the CTO. After passing through the CTO from a distal location, the distal end of the guidewire must be retrieved and passed outside the body so that a medical device (e.g., an atherectomy device, a CTO crossing device, an angioplasty balloon catheter, a stent, or another medical device) can be received on the guidewire and delivered to the treatment site.

### SUMMARY OF THE DISCLOSURE

In one aspect, a guidewire retrieval catheter generally comprises a guidewire retrieval lumen extending lengthwise of a catheter body and configured to receive a guidewire therein. An inflatable member at the distal end of the catheter body defines an inflation chamber for selectively receiving fluid to configure the inflatable member from deflated configuration to an inflated configuration. The inflatable member has an inner wall defining a guidewire retrieval cavity in communication with the guidewire retrieval lumen to facilitate insertion of the guidewire into the guidewire retrieval lumen when the inflatable member is in the inflated configuration.

In another aspect, a method of using the guidewire retrieval catheter to retrieve a guidewire is also disclosed.

Other features will be in part apparent and in part pointed out hereinafter.

### BRIEF DESCRIPTION OF THE DRAWINGS

FIG. 1 is a perspective of one embodiment of a guidewire retrieval catheter;

FIG. 2 is an enlarged, fragmentary longitudinal section of the guidewire retrieval catheter including a distal end thereof;

FIG. 3 is a schematic longitudinal section illustrating the guidewire retrieval catheter being introduced into an occluded body lumen, the guidewire retrieval catheter being in a deflated configuration;

FIG. 4 is similar to FIG. 3, except the guidewire retrieval catheter is in an inflated configured after being introduced into the occluded body lumen;

FIG. 5 is similar to FIG. 4, except a guidewire is being advanced through an occlusion in the body lumen toward the guidewire retrieval catheter;

FIG. 6 is similar to FIG. 5, except the guidewire is being captured within a guidewire retrieval cavity of an inflatable member of the guidewire retrieval catheter;

FIG. 7 is similar to FIG. 6, except the guidewire is being advanced within a guidewire retrieval lumen of the guidewire retrieval catheter toward a proximal end of the guidewire retrieval catheter;

FIG. 8 is an enlarged, fragmentary longitudinal section of another embodiment of a guidewire retrieval catheter including a distal end thereof, with a guidewire retention member being in a deflated configuration; and

FIG. 9 is similar to FIG. 8, except a guidewire is received in a guidewire retrieval lumen of the guidewire retrieval catheter, and the guidewire retention member is in an inflated configuration.

Corresponding reference characters indicate corresponding parts throughout the drawings.

### DETAILED DESCRIPTION OF THE DRAWINGS

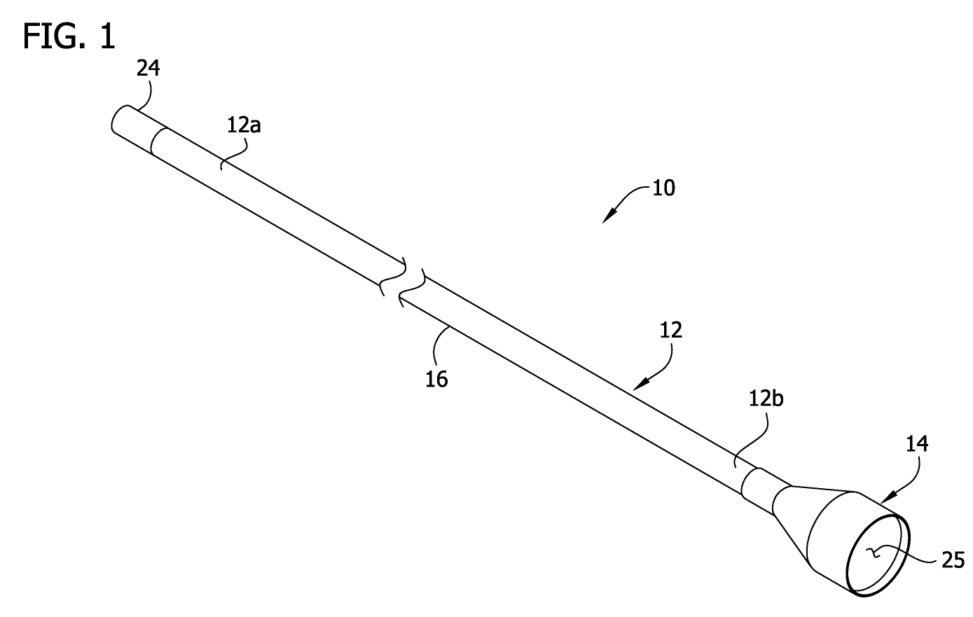

Referring to FIG. 1, a guidewire retrieval catheter for retrieving a guidewire disposed within a body lumen is generally indicated at reference numeral 10. As explained below, it is envisioned that the guidewire retrieval catheter 10 will be used during a medical procedure to facilitate retrieval of a retrograde guidewire that is introduced into the body lumen via a retrograde approach. The guidewire retrieval catheter 10 comprises a catheter body, generally indicated at reference numeral 12, and an inflatable member, generally indicated at reference numeral 14. The catheter body 12 has proximal and distal end portions 12a, 12b, and a length extending between the proximal and distal end portions. As explained in more detail below, the inflatable member 14 is disposed at the distal end portion 12b of the catheter body 12. The catheter body 12 has a suitable outer dimension for insertion into a desired body lumen, such as a peripheral artery of the leg, other blood vessels, or other body lumens. The catheter body 12 may be generally flexible to allow tracking through a tortious body lumen.

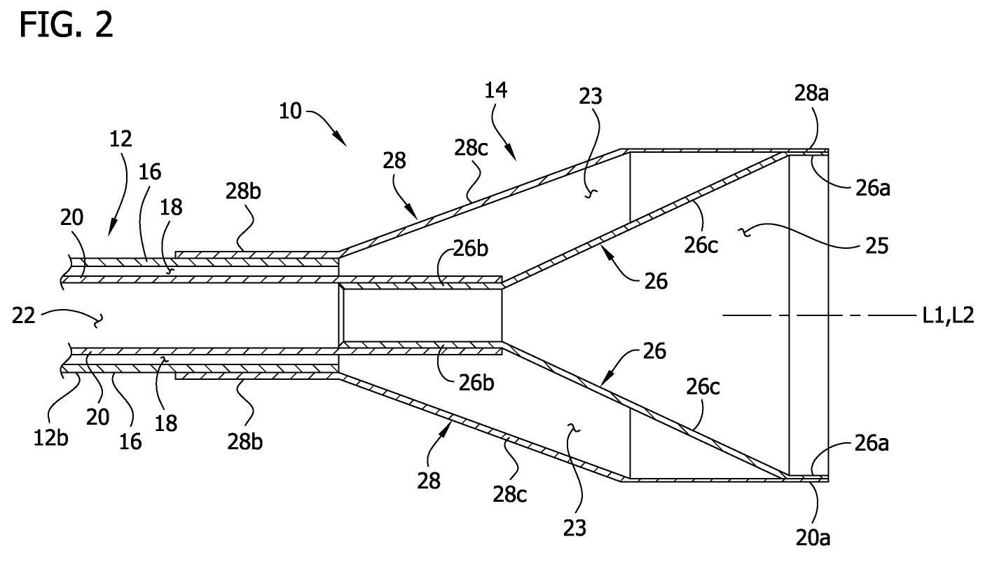

Referring to FIG. 2, the catheter body 12 comprises a first tubular member 16 (e.g., an outer tubular member) defining an inflation lumen 18 extending lengthwise of the catheter body in fluid communication with the inflatable member 14, and a second tubular member 20 defining a guidewire retrieval lumen 22 extending lengthwise of the catheter body and configured to receive a distal end portion of a retrograde guidewire therein. In the illustrated embodiment, the second tubular member 20 is disposed in the first tubular member 16, more specifically, within the inflation lumen 18 of the first tubular member. The illustrated tubular members 16, 20 are generally coaxial and concentric, and the first tubular member is considered a radially outer tubular member and the second tubular member is a radially inner tubular member. The outer and inner tubular members 16, 20 may be tack welded to one another at discrete locations. Also, the tubular members 16, 20 may be wall sections of a single tube defining the inflation lumen 18 and the guidewire retrieval lumen 22. The second tubular member 20 extends distally beyond the distal end of the first tubular member 16. It is understood that in other embodiments, the tubular members 16, 20 may not be coaxial and concentric. Moreover, in other embodiments the second tubular member 20 may not be received in the first tubular member 16. For example, the tubular members may extend side by side along the length of the catheter body 12 or the catheter body may have other configurations. Each of the tubular members 16, 20 may be formed from and/or comprise a polymeric material, such as, but not limited to, thermoplastic elastomer, nylon (e.g., nylon 12), polyurethane, polyethylene terephthalate, and blends thereof.

Referring still to FIG. 2, in the illustrated embodiment, the inflatable member 14 (e.g., a balloon) is secured to the distal end portion 12b of the catheter body 12, and in particular, the inflatable member is secured to each of the first and second tubular members 16, 20, respectively. A longitudinal axis L1 of the inflatable member 14 is generally coaxial with a longitudinal axis L2 of the catheter body 12. As explained in more detail below, the inflatable member 14 defines an inflation chamber 23 in fluid communication with the inflation lumen 18 of the first tubular member 16 for delivering fluid to the inflation chamber to inflate the inflation member (i.e., selectively configure the catheter in an inflated configuration) and for removing fluid from the inflation chamber to deflate the inflatable member (i.e., selectively configure the catheter in a deflated configuration). An inflation port 24 (FIG. 1) may be secured to the proximal end portion 12a of the catheter body 12 in fluid communication with the inflation lumen 18 for fluidly connecting a source of fluid (e.g., a syringe or other source) to the inflation lumen. As also explained in more detail below, the inflatable member 14 also defines a guidewire retrieval cavity 25 in communication with the guidewire retrieval lumen 22 to facilitate capturing of the retrograde guidewire in the guidewire retrieval lumen when the inflatable member is inflated.

The illustrated inflatable member 14 comprises an inner wall, generally indicated at 26, and an outer wall, generally indicated at 28, surrounding the inner wall. In the illustrated embodiment, the inner and outer walls 26, 28 are coaxial and generally concentric. The inner wall 26 has a distal end portion 26a secured (e.g., heat-welded, adhered, or otherwise attached) to a distal end portion 28a of the outer wall to form a fluid-tight, annular seal therebetween. A proximal end portion 26b of the inner wall 26 is secured (e.g., heat-welded, adhered, or otherwise attached) to (e.g., secured within) a distal end portion of the second tubular member 20 to form a fluid-tight, annular seal therebetween, and a proximal end portion 28b of the outer wall 28 is secured (e.g., secured to the outside of) a distal end portion of the first tubular member 16 to form a fluid-tight, annular seal therebetween. The inner and outer walls 26, 28 also have transition portions 26c, 28c, respectively, tapering proximally from the distal end portions 26a, 28a to the corresponding proximal end portions 26b, 28b. The inflation chamber 23 is defined radially between the inner and outer walls 26, 28, respectively, of the inflatable member 14 for receiving fluid to inflate the inflatable member 14.

At least a longitudinal portion of the inner wall 26 of the inflatable member 14 (e.g., the transition portion 26c in the illustrated embodiment) has an inner dimension defining a generally frustoconical shape of the guidewire retrieval cavity 25. This portion of the guidewire retrieval cavity 25 tapers proximally toward the distal end portion 12b of the catheter body 12 to facilitate insertion of the retrograde guidewire into the guidewire retrieval lumen 22. In one example, the inner and outer walls 26 of the inflatable member 14 may be formed from and/or comprise polyether block amide, nylon (e.g., nylon 12), urethane, polyethylene terephthalate, and blends thereof. The inner and outer walls 26, 28, respectively, may be formed from and/or comprise other materials without departing from the scope of the present invention. Because the distal end of the retrograde guidewire will likely contact and move along the inner wall 26 (more specifically the transition portion 26c of the inner wall) as the retrograde guidewire is fed into the guidewire retrieval cavity 25, the inner wall may comprise a material that has a Shore D hardness from about 25 to about 88 Shore D, such as about 60 Shore D, to inhibit puncturing of the inner wall by the guidewire. In one example, the Shore D hardness of the inner wall 26 may be greater than the Shore D hardness of the outer wall 28, thereby making the outer wall more compliant than the inner wall to facilitate conformance of the inflatable member 14 within the body lumen, for reasons explained in more detail below. This construction also facilitates refolding of the deflated inflatable member 14 after retrieval of the retrograde guidewire. As explained in more detail below, refolding of the inflatable member 14 when deflated facilitates removal of the guidewire retrieval catheter 10 from the body lumen.

The inflatable member 14 may be constructed by blow molding the inner and outer walls 26, 28. In an exemplary method, the inner and outer walls 26, 28 may be blow molded as a single structure having a shape and construction similar to an angioplasty balloon, with proximal and distal end portions. The single structure may be cut at a location(s) between the proximal and distal end portions to form the inner and outer walls 26, 28. The inner wall 26 can be inserted into the outer wall 28 and the distal ends of the walls attached to one another (e.g., laser bonding) to form the basic construct of the inflatable member 14. The balloon construct can be attached (e.g., laser bonding) to catheter body 12 by attaching the proximal end of the inner wall to the inner tubular member 20, and attaching (e.g., laser bonding) the proximal end of the outer wall to the outer tubular member 16. The inflatable member 14 is pleated and folded to reduce the outer dimension of the inflatable member. Other ways of constructing the inflatable member 14 do not depart from the scope of the present invention.

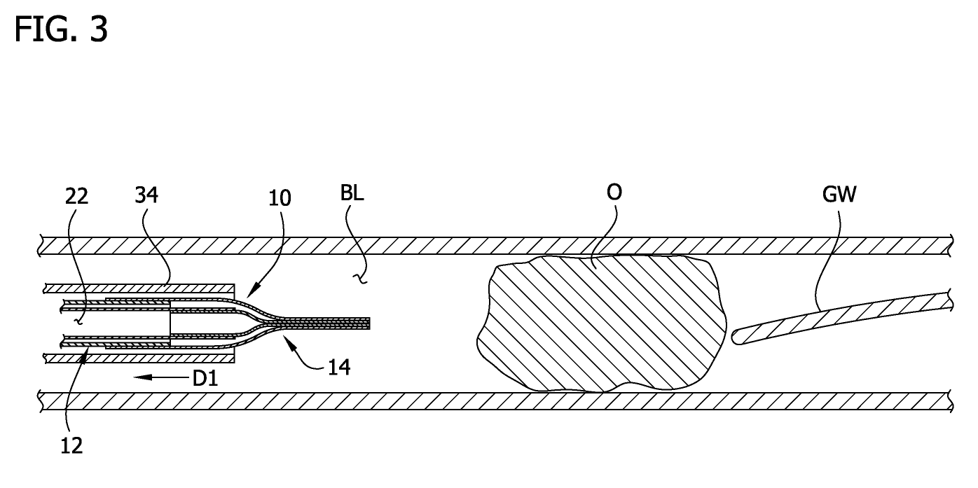

An exemplary method of retrieving a retrograde guidewire from a body lumen will now be described. Referring to FIG. 3, a retrograde guidewire GW is introduced into a body lumen BL at a first side (e.g., a distal side) of an occlusion O of the body lumen, and the guidewire retrieval catheter 10 is introduced into the body lumen at a second side (e.g., a proximal side) of the occlusion. For example, where the body lumen BL is a peripheral artery in a leg of a subject, the retrograde guidewire GW may be inserted into the artery at an inferior location so that the guidewire approaches a distal cap of the occlusion O, and the guidewire retrieval catheter 10 may be inserted into the artery at a superior location so that the guidewire retrieval catheter approaches the proximal cap of the occlusion. In one example, the occlusion O may be a chronic total occlusion.

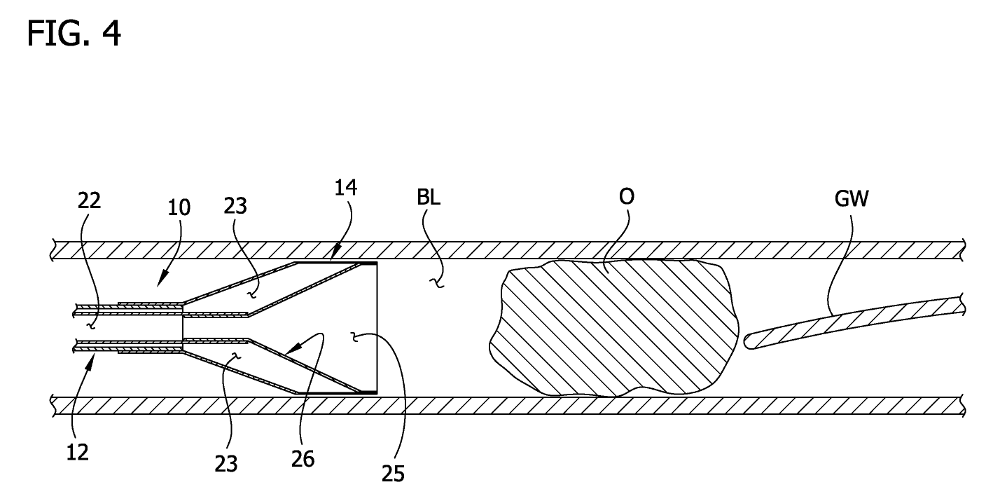

Referring still to FIG. 3, the guidewire retrieval catheter 10 may be inserted into the body lumen BL (e.g., a blood vessel) using an introducer sheath 34. In particular, the introducer sheath 34 may be delivered to a location adjacent the proximal cap of the occlusion O, and then the guidewire retrieval catheter 10 may be introduced into and fed along the introducer sheath to a distal end of the sheath. The introducer sheath 34 can then be withdrawn (e.g., pulled back in direction D1) from the body lumen BL leaving the distal end of the guidewire retrieval catheter 10 adjacent the proximal cap of the occlusion O. As shown in FIG. 3, the inflatable member 14 is pleated and folded to a reduced outer dimension to facilitate feeding of the guidewire retrieval catheter 10 into the introducer sheath 34. Marker bands (e.g., radiopaque bands, such as platinum or tantalum, not shown) may be on the distal end portion 12b of the catheter body 12 and/or on the inflatable member 14 to allow a user to confirm placement of the catheter 10 using imaging. With the guidewire retrieval catheter 10 at the desired location in the body lumen BL, fluid is introduced into the inflation lumen 18, such as by connecting a fluid source to the inflation port 24, to deliver the fluid into the inflation chamber 23 and inflate the inflatable member 14. The source of fluid may include liquid (e.g., saline) or gas (carbon dioxide). FIG. 4 shows the inflatable member 14 in its inflated configuration. Although FIGS. 3 and 4 show the guidewire retrieval catheter 10 being introduced and inflated before the retrograde guidewire GW has passed through the occlusion O, the guidewire retrieval catheter may be introduced and optionally inflated after the guidewire has passed through the occlusion or at other times during the procedure to retrieve the retrograde guidewire.

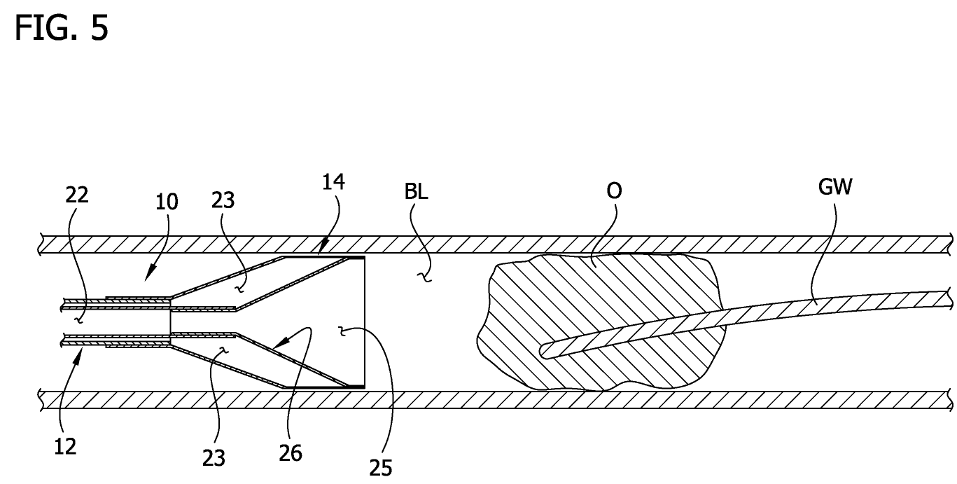

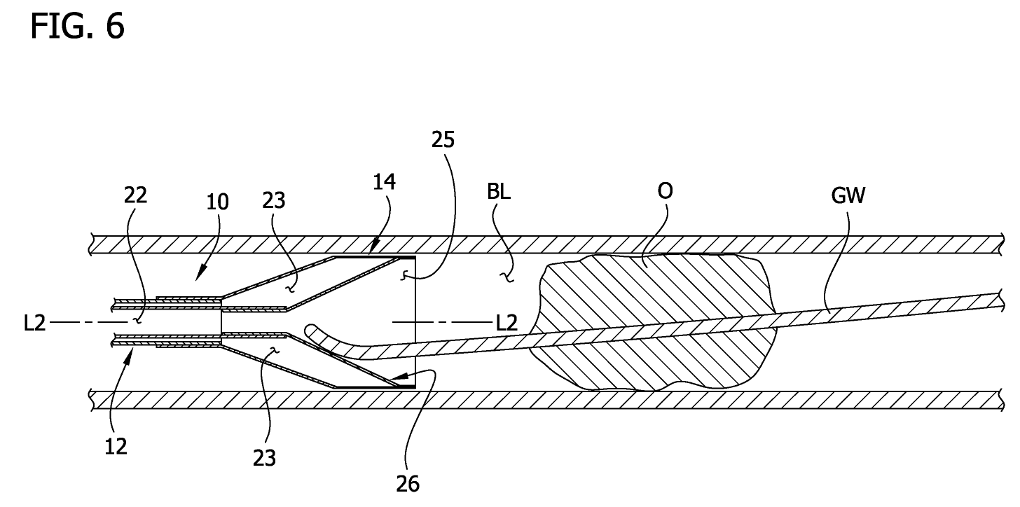

Referring to FIGS. 5 and 6, the retrograde guidewire GW is passed through the occlusion O from the distal cap through the proximal cap of the occlusion. After passing through the occlusion O, the guidewire GW is directed into the guidewire retrieval cavity 25. As can be seen from FIGS. 5 and 6, in its inflated configuration the inflatable member 14 has a cross-sectional dimension suitable for engaging the wall of the body lumen BL around substantially a 360 degree circumference of the body lumen wall. The outer wall 28 of the inflatable member 14 may generally conform to the shape of the body lumen wall to minimize any gaps between the outer wall of the inflatable member and the body lumen wall. Moreover, the opening of the guidewire retrieval cavity 25 may have generally the same inner cross-sectional dimension as the body lumen BL. In this way, the inflatable member 14 inhibits the guidewire GW from passing between the inflatable member and the body lumen wall and facilitates capturing the retrograde guidewire in the guidewire retrieval cavity 25.

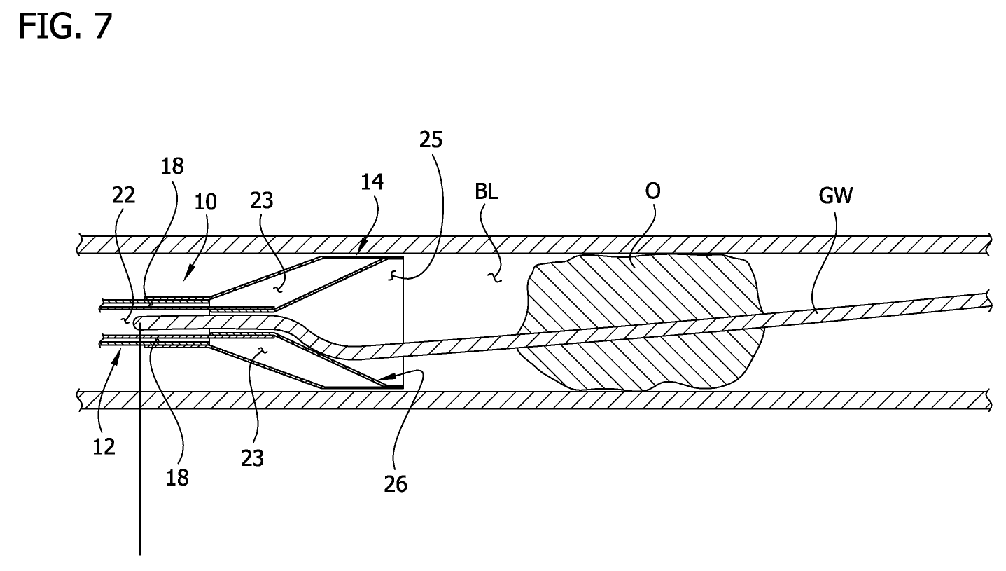

As shown in FIGS. 6, if the tip (i.e., the distal end) of the guidewire GW is offset from the axis L2 of the guidewire retrieval lumen 22, the guidewire tip will contact the frustoconical-shaped transition portion 26c of the inner wall 26 of the inflatable member 14. As described above, the inner wall 26 may be formed from a material suitable for inhibiting puncturing by the tip of guidewire GW. As shown in FIGS. 6 and 7, the frustoconical-shaped transition portion 26c of the inner wall 26 directs the tip of the retrograde guidewire toward and into the guidewire retrieval lumen 22 as the guidewire tip continues to advance in the guidewire retrieval cavity 25. In other words, the guidewire tip is funneled toward and into the guidewire retrieval lumen 22 by the frustoconical-shaped transition portion 26c as the guidewire is advanced. As shown in FIG. 7, the guidewire GW is fed through the guidewire retrieval lumen 22, in a proximal direction relative to the guidewire retrieval catheter 10, and exits the guidewire retrieval lumen (and the catheter 10) at a location outside the subject's body (not shown). With the tip of the retrograde guidewire GW disposed outside the subject's body, the guidewire retrieval catheter 10 can be removed from the body lumen BL. In particular, the inflatable member 14 is first deflated, such as by applying negative pressure to the inflation port 24 to withdraw the fluid from the inflation chamber 23 through the inflation lumen 18. Then the deflated guidewire retrieval catheter 10 can be pulled back (e.g., moved proximally) into the distal end of the introducer sheath 34. The compliant outer layer 28 may facilitate re-folding of the inflatable member 14 to facilitate pull-back into the introducer sheath 34.

After removing the guidewire retrieval catheter 10, a medical device (e.g., a directional atherectomy device, a CTO crosser, a balloon catheter, etc.) can be inserted into the body lumen BL using the retrograde guidewire GW. The medical device can be fed along the retrograde guidewire GW to the proximal cap of the occlusion O to treat the occlusion. It is understood that the steps recited above may be modified, omitted, or performed any a different order for retrieving a retrograde guidewire in a body lumen.

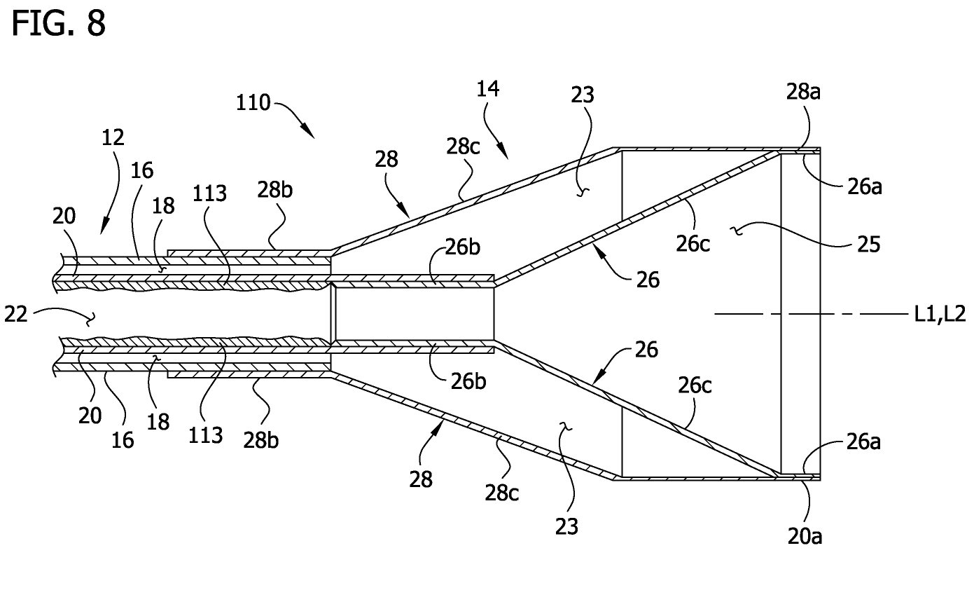

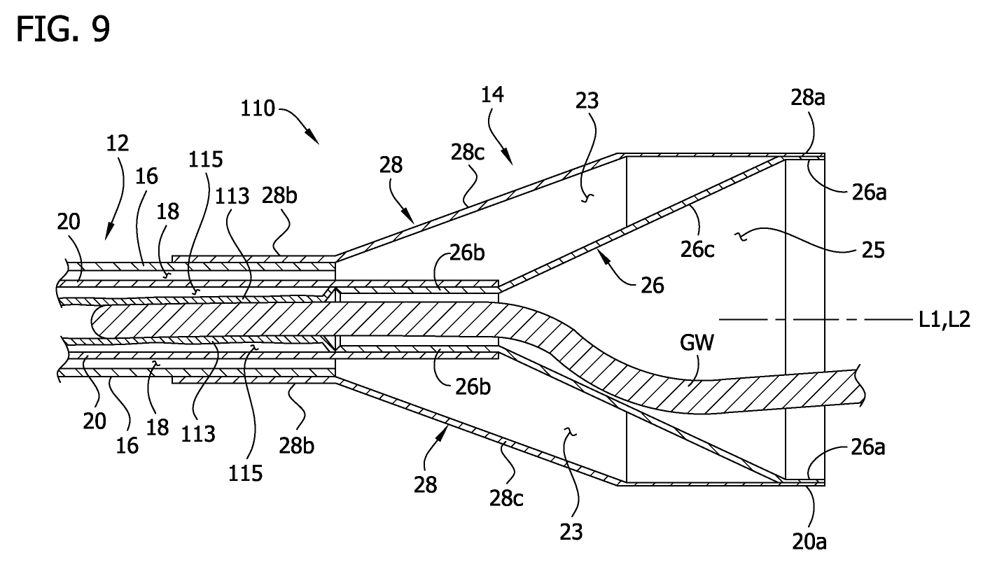

Referring to FIGS. 8 and 9, another guidewire retrieval catheter is generally indicated at reference numeral 110. Other than the difference(s) described below, this catheter 110 is substantially identical to the first catheter 10. Accordingly, identical components are indicated by corresponding reference numerals. Unlike the first catheter 10, this catheter 110 includes a guidewire retention member 113 configured to grip a retrograde guidewire GW in the guidewire retrieval lumen 22 so that the guidewire moves with the guidewire retrieval catheter as the guidewire retrieval catheter is withdrawn from a body lumen. Thus, as explained below, rather than feeding the tip of the retrograde guidewire GW through the guidewire retrieval lumen 22 to outside the body lumen and the subject's body, the tip of the guidewire is removed from the body lumen with the guidewire retrieval catheter. In the illustrated embodiment, the guidewire retention member 113 is inflatable (broadly, a second inflatable member) and disposed inside the guidewire retrieval lumen 22 such that the guidewire retention member expands radially inward within the guidewire retrieval lumen when inflated with a fluid. In particular, the inflatable retention member 113 includes a wall secured to the inner surface of the second tubular member 20 to define an inflatable lumen 115 (FIG. 9) therebetween. A source of fluid (not shown) is connectable to a proximal end (not shown) of the catheter 110 for delivering fluid into the inflatable lumen 115 to radially expand the retention member toward the longitudinal axis L2 of the second tubular member 20. In the illustrated embodiment, the inflatable retention member 113 has a generally annular cross-sectional shape, although it may be of other shapes. The guidewire retention member 113 may be of other configurations for gripping the guidewire GW in the guidewire retrieval lumen 22.

An exemplary method of retrieving a retrograde guidewire from a body lumen using the guidewire retrieval catheter 110 will now be described. The steps performed for retrieving the retrograde guidewire GW using the catheter 110 are the same as the steps described above for the first catheter 10, other than the steps for removing the retrograde guidewire from the body lumen (not shown). After (or before) the tip of the catheter 110 is inserted into the guidewire retrieval lumen 22, fluid is delivered into the inflatable retention lumen 115 of the inflatable retention member 113 to radially expand the retention member toward the longitudinal axis L2 of the second tubular member 20. As the inflatable retention member 115 expands, it engages the portion of the guidewire GW received in the guidewire retrieval lumen 22 (e.g., the distal end portion of the guidewire) and retains the guidewire in the lumen by an interference fit. With the guidewire GW retained in the lumen 22, the inflatable member 14 is deflated, and the catheter 110 and the guidewire are withdrawn from the body lumen. As an example, the catheter 110 may be re-sheathed (e.g., pulled back) into the introducer sheath (or other catheter) to remove it, along with the guidewire GW, from the body lumen. Once outside the body, the inflatable retention member 113 can be deflated and the tip of the guidewire GW can be removed (e.g., withdrawn) from the catheter 110. It is understood that the steps recited above may be modified, omitted, or performed any a different order for retrieving a retrograde guidewire in a body lumen.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A guidewire retrieval catheter comprising:
a catheter body having proximal and distal ends, a length extending between the proximal and distal ends, and a guidewire retrieval lumen extending lengthwise of the catheter body and configured to receive a guidewire therein;
an inflatable member at the distal end of the catheter body, the inflatable member defining an inflation chamber for selectively receiving fluid to configure the inflatable member from deflated configuration to an inflated configuration, wherein the inflatable member has an inner wall defining a guidewire retrieval cavity in communication with the guidewire retrieval lumen to facilitate insertion of the guidewire into the guidewire retrieval lumen when the inflatable member is in the inflated configuration.

2. The guidewire retrieval catheter set forth in claim 1, wherein at least a portion of the inner wall has an inner dimension tapering proximally toward the guidewire retrieval lumen when the inflatable member is in the inflated configuration.

3. The guidewire retrieval catheter set forth in claim 2, wherein the inflatable member has an outer wall surrounding the inner wall, wherein a space between the inner and outer walls defines at least a portion of the inflation chamber.

4. The guidewire retrieval catheter set forth in claim 3, wherein proximal end portions of the inner and outer walls of the inflatable member are secured to the catheter body.

5. The guidewire retrieval catheter set forth in claim 4, wherein distal end portions of the inner and outer walls of the inflatable member are secured to one another.

6. The guidewire retrieval catheter set forth in claim 1, wherein the catheter body includes a first tubular member defining an inflation lumen in fluid communication with the inflation chamber, and a second tubular member defining the guidewire retrieval lumen.

7. The guidewire retrieval catheter set forth in claim 6, wherein the second tubular member is disposed in the first tubular member.

8. The guidewire retrieval catheter set forth in claim 7, wherein the inflatable member has an outer wall surrounding the inner wall, the inner wall of the inflatable member having a proximal end portion secured to the first tubular member, the outer wall of the inflatable member having a proximal end portion secured to the second tubular member.

9. The guidewire retrieval catheter set forth in claim 6, wherein the second tubular member has a distal end portion extending distally outward relative to a distal end of the first tubular member.

10. The guidewire retrieval catheter set forth in claim 1, wherein the inflatable member has an outer wall surrounding the inner wall, wherein a radial space between the inner and outer walls defines at least a portion of the inflation chamber.

11. The guidewire retrieval catheter set forth in claim 10, wherein the inner and outer walls of the inflatable member comprises polymeric material.

12. The guidewire retrieval catheter set forth in claim 11, wherein a Shore D hardness of the inner wall is greater than a Shore D hardness of the outer wall.

13. The guidewire retrieval catheter set forth in claim 1, wherein an outer maximum dimension of the inflatable member is expanded when the inflatable member is in the inflatable configuration.

14. The guidewire retrieval catheter set forth in claim 1, wherein the inflatable member extends distally outward from the distal end of the catheter body.

15. The guidewire retrieval catheter set forth in claim 1, wherein at least a portion of the inner wall of the inflatable member has a generally frustoconical shape.

16. The guidewire retrieval catheter set forth in claim 1, further comprising a second inflatable member disposed in the guidewire retrieval lumen, the second inflatable member defining a second inflation chamber for selectively receiving fluid to configure the second inflatable member from deflated configuration to an inflated configuration, wherein the second inflatable member is configured to grip the guidewire in the guidewire retrieval lumen when the second inflatable member is in the inflated configuration.

17. The guidewire retrieval catheter set forth in claim 16, wherein the inflatable member and the second inflatable member are configurable between respective deflated and inflated configurations independent of one another.

18. A method of retrieving a guidewire from a body lumen, the method comprising:
inserting a distal end of a guidewire retrieval catheter into a body lumen of a subject, wherein the guidewire retrieval catheter includes a catheter body having a guidewire retrieval lumen configured to receive a guidewire therein, and an inflatable member at a distal end of the catheter;
delivering, after said inserting a distal end of a guidewire retrieval catheter, fluid into an inflation chamber of the inflatable member to inflate the inflatable member so that an inner wall of the inflatable member defines a guidewire retrieval cavity in communication with the guidewire retrieval lumen;
inserting, after said delivering fluid into an inflation chamber, a distal end of a guidewire disposed in the body lumen through the guidewire retrieval cavity of the inflatable member and into the guidewire retrieval lumen of the catheter body.

19. The method of retrieving a guidewire from a body lumen set forth in claim 18, further comprising feeding, after said inserting a distal end of a guidewire, the distal end of the guidewire through the guidewire retrieval lumen to outside the body of the subject.

20. The method of retrieving a guidewire from a body lumen set forth in claim 18, further comprising inflating, after said inserting a distal end of a guidewire, a second inflatable member within the guidewire retrieval lumen of the catheter body to retain the distal end of the guidewire in the guidewire retrieval lumen.
